(19) United States
(12) Patent Application Publication
(10) Pub. No.: US 2004/0092428 A1
(43) Pub. Date: May 13, 2004

(54) Oral pharmaceuticals formulation comprising paclitaxel, derivatives and methods of administration thereof

(76) Inventors: Hongming Chen, Acton, MA (US); Zhong Zhang, Sudbury, MA (US); Szu-Wen Wang, Irvine, CA (US)

Correspondence Address: TRANSFORM PHARMACEUTICALS, INC., 29 HARTWELL AVENUE, LEXINGTON, MA, 02421, US

(21) Appl. No.: 10/344,018

(22) PCT Filed: Nov. 26, 2002
(86) PCT No.: PCT/US02/37707

(30) Foreign Application Priority Data
Nov. 27, 2001 (US) 60333999
Sep. 19, 2002 (US) 60412675

(51) Int. Cl. A61K 38/00

(52) U.S. Cl. 514/2

(57) ABSTRACT

The invention concerns excipients or combinations thereof suitable for preparing an oral formulation containing a pharmaceutical agent. More particularly, the invention is directed to stable, efficacious and bioavailable oral pharmaceutical formulations comprising paclitaxel, derivatives of paclitaxel and pharmaceutically acceptable salts thereof. The formulations of the invention increase bioavailability of paclitaxel when dissolved in the gastrointestinal system. The formulations of the invention are useful for administering paclitaxel, its derivatives, or pharmaceutically acceptable salts of such derivatives to patients in need thereof. The formulations of the invention are particularly suitable for oral administration to mammals including humans.

### 1. FIELD OF THE INVENTION

The present invention is directed to excipients or combinations thereof suitable for preparing an oral formulation containing a pharmaceutical agent. More particularly, the invention is directed to stable, efficacious and bioavailable pharmaceutical formulations used to orally deliver paclitaxel, derivatives of paclitaxel or pharmaceutically acceptable salts thereof to human patients. Finally, the invention relates to combination therapy to improve the bioavailability of anti-cancer agents

### 2. BACKGROUND OF THE INVENTION

### 2.1. Pharmaceutical Formulations

Pharmaceuticals are rarely distributed or administered as pure compounds because of problems with, among others, stability, solubility, and bioavailability of the pharmaceutical itself (i.e., the active). In most cases, pharmaceuticals are administered in a pharmaceutical formulation comprising the active, and other components, such as excipients, binders, diluents, and other delivery vehicles, carriers or systems. It is well documented that physical and chemical properties, such as stability, solubility, dissolution, permeability, and partitioning of most pharmaceuticals are directly related to the medium in which they are administered. And, in turn, the physical and chemical properties of drug-in-formulation mixtures affect the pharmacological and pharmacokinetic properties, such as absorption, bioavailability, metabolic profile, toxicity, and potency. Such effects are caused by interactions between the formulation's components and the pharmaceutical and/or interactions between the components themselves. Other properties influenced by the formulation in which a pharmaceutical is administered include mechanical properties, such as compressibility, compactability, and flow characteristics and sensory properties, such as taste, smell and color. Thus, discovery of pharmaceutical formulations that optimize bioavailability and duration of action of the pharmaceutical and minimize undesirable properties is an important part of pharmaceutical development and research. For a general review of the subject of formulations see Howard, Introduction to Pharmaceutical Dosage Forms, Lea & Febiger, Philadelphia Pa., 4^{th} ed., 1985; Remington: the Science and Practice of Pharmacy, Alfonso R. Gennaro ed., Mack Publishing Co. Easton, Pa., 19th ed., 1995, Chapter 83.

### 2.2. Oral Bioavailability

In general, an oral formulation is preferred because of the distinct advantages over other methods of administration, in particular intravenous administration. In addition to the flexibility of treatment, the higher rate of compliance and the convenience and ease of administration associated with the elimination of hospital and physician supervision, oral formulations often create a substantial cost savings. However, many pharmaceutical formulations cannot be effectively administered orally because of the poor bioavailability of the active compounds as a result of low absorption from the gastrointestinal tract. Consequently, these active ingredients tend to be administered intravenously or intramuscularly. One such active ingredient that is not administered orally due to its poor bioavailability is paclitaxel.

Further, bioavailability is affected by cell surface proteins present in the gastrointestinal system, such as P-glycoproteins which can reduce gastrointestinal absorption of compounds in the gastrointestinal system. More specifically, they can prevent certain pharmaceutical active ingredients from being passed through the mucosal cells of the small intestines. As a result, the active ingredient is prevented from entering the bloodstream and is thus unable to be utilized. There is a need, therefore, for formulations that can orally deliver compounds, such as paclitaxel, despite its poor solubility, stability and bioavailability and despite the activity of P-glycoproteins.

Further evidence suggests that particle size plays a significant role in the absorption of a pharmaceutical ingredient into the gastrointestinal tract. As such, there is a need for a paclitaxel formulation which upon dilution into the gastric fluid remains dissolved or maintains a sufficiently small particle size.

### 2.3. Paclitaxel

Paclitaxel is presently available in the United States only as a non-aqueous sub-optimal formulation concentrate for intravenous injection. An intravenous dosage regimen of 135 mg/m² paclitaxel is recommended for previously untreated patients with carcinoma of the ovary, given every three weeks. Similar dosage regimens are recommend for other carcinomas. Paclitaxel is practically insoluble in water. The commercially-available paclitaxel formulation (Bristol-Myers Squibb) comprises 6 mg/ml of paclitaxel dissolved in Cremophor® EL (castor oil, polyoxyethylated castor oil) and dehydrated ethanol (50% v/v). Similar formulations are sold by other manufacturers, for example, IVAX Co. Before intravenous injection, the commercial dose must be diluted to a final concentration of 0.3 to 1.2 mg/ml prior to injection. Recommended diluents are 0.9% aqueous sodium chloride, 5% aqueous dextrose, or 0.9% sodium chloride 5% dextrose aqueous solution, or 5% dextrose in Ringer's injection (The Physician's Desk Reference, 54th edition, 881-887, Medical Economics Company (2000); Goldspiel 1994 Ann. Pharmacotherapy 28:S23-26, both of which are incorporated herein by reference). There is currently no commercially available oral formulation of paclitaxel.

In general, the toxicity of Cremophor® EL, which is a required component of the intravenous formulation, is believed to be unacceptable. There is a need, therefore, for formulations comprising paclitaxel, derivatives and pharmaceutically acceptable salts thereof that can deliver therapeutically effective amounts of paclitaxel and derivatives thereof that overcome the disadvantages caused by paclitaxel's insolubility and the disadvantages of intravenous delivery.

Current formulations, e.g. 6 mg/mL of paclitaxel in Ethanol and Cremophor®EL, are further disadvantageous because of the large dosage volumes required due to the low concentration of paclitaxel. Furthermore, recent studies have suggested that Cremophor®EL binds to paclitaxel, entrapping it in micelles, and decreasing absorption from the gut. (Bardelmeijer et al. Cancer Chemother. Pharmacol. 2002, 49:119-125; Malingré et. al. British Journal of Cancer 2001, 85(10), 1472-1477.) Thus, the bioavailability of paclitaxel is quite low when administered orally. There is a need, therefor, for formulations comprising paclitaxel, derivatives and pharmaceutically acceptable salts thereof which have an increased dissolution rate and uptake in the gastrointestinal system and hence a greater bioavailability for oral administration.

In addition, paclitaxel acts a substrate of P-glycoprotein. As a result, the oral bioavailability of paclitaxel is quite limited. Sparreboom et al. Proc. Natl. Acad. Sci., vol. 94, pp 2031-2035, 1997. There is a need, therefor, for formulations comprising paclitaxel, derivatives, and pharmaceutically acceptable salts thereof that can deliver therapeutically effective amounts of pactitaxel and derivatives thereof in a stable oral formulation with an increased bioavailability.

### 3. SUMMARY OF THE INVENTION

The invention encompasses oral paclitaxel formulations which are both stable during storage and have increased bioavailability. The formulations of the invention are useful for orally administering paclitaxel, its derivatives, or pharmaceutically acceptable salts of such derivatives to patients in need thereof. The formulations of the invention are particularly useful for increasing the oral bioavailability of paclitaxel, such that the oral route is a useful route of administration.

More generally, the formulations of the invention or mixtures thereof can increase the solubility of paclitaxel and derivatives thereof as well as its absorption in the gastrointestinal system such that the invention encompasses an oral formulation which can be used without relying on costly, inconvenient and potentially toxic intravenous formulations and administration.

In one embodiment, the invention concerns a pharmaceutical formulation suitable for oral administration to a mammal comprising:

(a) paclitaxel, a derivative, or a pharmaceutically acceptable salt thereof; and

(b) one or more of an oil, solvent, or surfactant each of which is defined further below.

In another embodiment, the invention concerns a pharmaceutical formulation suitable for oral administration to a mammal comprising:

(a) paclitaxel, a derivative, or a pharmaceutically acceptable salt thereof; and

(b) an oil, a solvent, and a surfactant.

In a further embodiment of the invention, the pharmaceutical formulations further comprise an acid to maintain solution stability; preferably an organic acid, such as citric acid.

In another embodiment, the pharmaceutical formulations of the invention are ethanol-free. In still another embodiment, the pharmaceutical formulations of the invention do not comprise cremophor.

In a preferred embodiment of the invention, the pharmaceutical formulation is suitable for oral administration via: capsules, cachets, soft elastic gelatin capsules, hard gelatin capsules, tablets, caplets; as aerosols sprays; as a powder or granules; as a solution or a suspension in a non-aqueous liquid, an oil-in-water emulsion, or a water-in-oil liquid emulsion each containing a predetermined amount of the active ingredients.

The invention encompasses oral paclitaxel formulations which have higher solubilities of paclitaxel over previous attempts to make oral paclitaxel. The formulations of the invention provide for a higher concentration of paclitaxel while lowering the overall volume of the dose of paclitaxel needed for therapeutic effect.

The invention encompasses oral paclitaxel formulations which have improved bioavailability over previous attempts to make an oral paclitaxel formulation. The formulations of the invention are absorbed by the cells of the gastrointestinal tract after dissolution and passage through the gastric fluid. The invention encompasses formulations that precipitate in the gastrointestinal system into particles of a size suitable for an increase in the dissloution profile of paclitaxel. In a related embodiment, formulations of the invention remain dissolved in gastric fluid for absorption, or preferably precipitate into discrete particles which measure less than about 10 μm in size; that is, the particles are of a size which increases the solubility and dissolution rate of paclitaxel in the gastrointestinal system. Preferably, the formulation has a particle size of less than about 5 μm in size, more preferably less than about 1 μm in size, and still more preferably less than about 600 nm in size.

The invention also encompasses processes for preparing oral formulations of paclitaxel or derivatives thereof. In one process, the formulations of the invention are prepared by dissolving paclitaxel, a derivative or a pharmaceutically acceptable salt thereof in a solvent of the invention prior to dilution with one or more paclitaxel-free oils, solvents, or surfactants as described herein. Such a method increases the amount of paclitaxel that can be formulated and thus delivered orally, as well as affects the particle size of the precipitate that forms upon contact with gastric fluid and gastrointestinal media. In a preferred embodiment, the solvent used in this process is PEG-400 or Transcutol or mixtures thereof. In another preferred embodiment, the concentration of paclitaxel after dilution with one or more paclitaxel-free oils, solvents, or surfactants is 100 mg/mL. In still another preferred embodiment, said paclitaxel-free oils, solvents, or surfactants include, but are not limited to, Triacetin, Transcutol or polysorbate 80.

The invention further encompasses methods of orally delivering paclitaxel to a mammal which comprises administering paclitaxel within one of the formulations of the invention. In a preferred embodiment, the invention encompasses methods of augmenting the bioavailability of paclitaxel in a mammal; more preferably increasing the oral bioavailability of paclitaxel in a mammal.

Further, the invention also encompasses methods of orally delivering paclitaxel to a mammal which comprises administering paclitaxel adjunctively with one or more inhibitors of P-glycoprotein. In other words, before, during, or after the administration of paclitaxel or a pharmaceutically acceptable derivative thereof, an inhibitor of P-glycoprotein is also administered such that the uptake of paclitaxel by the gastrointestinal system is improved. In a preferred embodiment, the P-glycoprotein inhibitor is administered as part of the oral formulation. In another embodiment, the paclitaxel and the p-glycoprotein inhibitor are administered separately but adjunctively, that is concurrently or sequentially. In another embodiment, the invention encompasses methods of orally delivering paclitaxel to a mammal which comprises administering paclitaxel adjunctively with one or more inhibitors of P-glycoprotein wherein the P-glycoprotein is administered in an amount less than previously found effective.

### 3.1. BRIEF DESCRIPTION OF THE FIGURES

FIG. 1. Pharmacokinetic profiles for Formulations 1 (83 mg/mL paclitaxel dissolved in 4.9% Triacetin, 34.0% PEG 400, 58.0% Polysorbate 80, 3.0% water) and Formulation 2 (100 mg/mL paclitaxel dissolved in 75% Transcutol, and 25% vitamin E TPGS)alone and with or without a p-glycoprotein inhibitor cyclosporin A (CsA).

### 3.2. Definitions

The term “mammal” as used herein, encompasses any mammal. Preferably a mammal is in need of a formulation of the invention. Examples of mammals include, but are not limited to, cows, horses, sheep, pigs, cats, dogs, mice, rats, rabbits, guinea pigs, monkeys, etc., more preferably, a human. In certain embodiments, the mammal is an infant, child, adolescent or adult.

As referred to herein, derivatives and analogs of paclitaxel include, but are not limited to, docetaxel and compounds having the general formula I below and stereoisomers and pharmaceutically acceptable salts thereof:

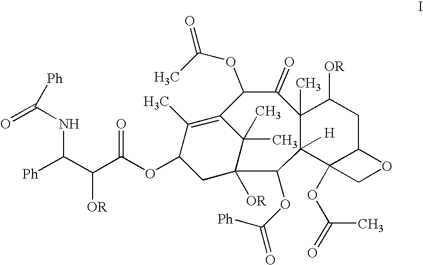

wherein, each occurrence of R is independently H, (C₁-C₆)alkyl, aryl, C(O)(C₁-C₆)alkyl, or C(O)aryl. Preferably, (C₁-C₆)alkyl is methyl and aryl is phenyl. Such derivatives are well known in the art. For example, paclitaxel derivatives encompassed by formula I are disclosed in U.S. Pat. Nos. 5,399,726; 5,654,447; 6,066,747; 5,338,872; 6,107,332; 5,703,117; 5,714,512; 5,580,899; 6,118,011; 5,470,866; 5,319,112; and 6,136,961.

As used herein, the term “alkyl group” means a saturated, monovalent, unbranched or branched hydrocarbon chain. Examples of alkyl groups include, but are not limited to, (C₁-C₂₅)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-proyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, and longer alkyl groups, such as heptyl, and octyl, nonyl, decyl, undecyl, dodecyl, tridecyl, tetradecyl, pentadecyl, hexadecyl, heptadecyl, octadecyl, nonadecyl, icosanyl, henicosanyl, docosanyl, tricosanyl, tetracosanyl, and pentacosanyl. An alkyl group can be unsubstituted or substituted with one or more suitable substituents.

An “alkenyl group” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₂₅)alkenyl groups, such as vinyl, allyl, butenyl, pentenyl, hexenyl, butadienyl, pentadienyl, hexadienyl, 2-ethylhexyl, 2-propyl-2-butenyl, 4-(2-methyl-3-butene)-pentenyl, nonenyl, decenyl, undecenyl, dodecenyl, tridecenyl, tetradecenyl, pentadecenyl, hexadecenyl, heptadecenyl, octadecenyl, nonadecenyl, icosanenyl, henicosanenyl, docosanenyl, tricosanenyl, tetracosanenyl, and pentacosanenyl. An alkenyl group can be unsubstituted or substituted with one or more suitable substituents.

An “alkynyl group” means monovalent, unbranched or branched hydrocarbon chain having one or more triple bonds therein. The triple bond of an alkynyl group can be unconjugated or conjugated to another unsaturated group. Suitable alkynyl groups include, but are not limited to, (C₂-C₂₅)alkynyl groups, such as ethynyl, propynyl, butynyl, pentynyl, hexynyl, methylpropynyl, 4-methyl-1-butynyl, 4-propyl-2-pentynyl, 4-butyl-2-hexynyl, nonynyl, decynyl, undecynyl, dodecynyl, tridecynyl, tetradecynyl, pentadecynyl, hexadecenyl, heptadecynyl, octadecynyl, nonadecynyl, icosanynyl, henicosanynyl, docosanynyl, tricosanynyl, tetracosanynyl, and pentacosanynyl. An alkynyl group can be unsubstituted or substituted with one or more suitable substituents.

An “aryl group” means a monocyclic or polycyclic-aromatic ring comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more suitable substituents. Preferably, the aryl group is a monocyclic ring, wherein the ring comprises 6 carbon atoms, referred to herein as “(C₆)aryl”.

A “cycloalkyl group” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or more suitable substituents. Preferably, the cycloalkyl group is a monocyclic ring or bicyclic ring.

The term “alkoxy group” means an —O-alkyl group, wherein alkyl is as defined above. An alkoxy group can be unsubstituted or substituted with one or more suitable substituents. Preferably, the alkyl chain of an alkoxy group is from 1 to 25 carbon atoms in length, referred to herein as “—(C₁-C₂₅)alkoxy”.

The term “aryloxy group” means an —O-aryl group, wherein aryl is as defined above. An aryloxy group can be unsubstituted or substituted with one or more suitable substituents. Preferably, the aryl ring of an aryloxy group is a monocyclic ring, wherein the ring comprises 6 carbon atoms, referred to herein as “(C₆)aryloxy”.

The term “benzyl” means —CH₂-phenyl.

The term “phenyl” means —C₆H₅. A phenyl group can be unsubstituted or substituted with one or more suitable substituents.

A “carbonyl” group is a divalent group of the formula —C(O)—.

An “alkoxycarbonyl” group means a monovalent group of the formula —C(O)-alkoxy. Preferably, the hydrocarbon chain of an alkoxycarbonyl group is from 1 to 25 carbon atoms in length.

As used herein, “halogen” means fluorine, chlorine, bromine, or iodine. Correspondingly, the meaning of the terms “halo” and “Hal” encompass fluoro, chloro, bromo, and iodo.

As used herein, a “suitable substituent” means a group that does not nullify the synthetic or pharmaceutical utility of the active or the paclitaxel solubilizer of the invention. Examples of suitable substituents include, but are not limited to: (C₁-C₈)alkyl; (C₁-C₈)alkenyl; (C₁-C₈)alkynyl; (C₆)aryl; (C₂-C₅)heteroaryl; (C₃-C₇)cycloalkyl; (C₁-C₈)alkoxy; (C₆)aryloxy; CN; OH; oxo; halo, CO₂H; NH₂; NH((C₁-C₈)alkyl); N((C₁-C₈)alkyl)₂; NH((C₆)aryl); N((C₆)aryl)₂; CHO; CO((C₁-C₈)alkyl); CO((C₆)aryl); CO₂((C₁-C₈)alkyl); and CO₂((C₆)aryl). One of skill in art can readily choose a suitable substituent based on the stability and pharmacological and synthetic activity of the paclitaxel solubilizer of the invention.

As used herein, the terms “oil” and “oil of the invention” refer to a compound including, but not limited to, Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil--hydrogenated soybean oil, Captex 335 (C8/C10 triglycerides from coconut oil), corn glycerides, corn oil, corn oil PEG-6 esters, cottonseed oil, Captex 200 (C8/C10 diesters of propylene glycol of coconut oil), diacetylated monoglycerides, Sesame oil, Soybean oil hydrogenated, Capmul MCM (C8/C10 mono-/diglycerides from coconut oil), Benzyl Benzoate, Soybean oil, olive oil, PEG vegetable oil, Vegetable oil, Vegetable oil hydrogenated, peanut oil, mineral oil, or Vegetable shortening.

As used herein, the terms “solvent” and “solvent of the invention” refer to a compound including, but not limited to, ethanol, cetyl alcohol, glyceryl stearate, isopropyl alcohol, diethylamine, ethylene glycol monoethyl ether, Transcutol, benzyl alcohol, glyceryl oleate, gelucire, myristyl alcohol, diethanolamine, glycerin, glyceryl distearate, gamma cyclodextrin, gelatin, ethylene glycol, polyethylene glycol 8000, Cresol, Propylene glycol, polyethylene glycol, polyethylene glycol 1000, polyethylene glycol 1450, polyethylene glycol 1540, polyethylene glycol 200, polyethylene glycol 300, polyethylene glycol 3350, polyethylene glycol 3500, Povidone, polyethylene glycol 400, polyethylene glycol 4000, polyethylene glycol 600, polyethylene glycol 6000, Stearyl alcohol, polyethylene glycol t-dodecylthioehter, polyethylene oxide, Triacetin, Polyvinylpyridine, Polyvinyl alcohol, Polypropylene glycol, or Arlacel 186 (monoolein:propylene glycol=90:10)

As used herein, the terms “surfactant” and “surfactant of the invention” refer to a compound including, but not limited to, Polyoxyl 20 stearate, Polyoxyl 35 castor oil, poloxamer, polyoxyethylene sorbitan monoisostearate, polyethylene glycol 40 sorbitan diisostearate, Polyoxyl 40 Hydrogenated castor oil, Polysorbate, Polysorbate 20, Polysorbate 40, Polyoxyl 60 stearate, Polysorbate 85, Polysorbate60, poloxamer 331, polyoxyethylene fatty acid esters, Polyoxyl 40 castor oil, poloxamer 188, polyoxyethylene polyoxypropylene 1800, oleic acid, Sodium desoxycholate, Sodium lauryl sulfate, Sorbitan monolaurate, Sorbitan monooleate, Sorbitan monopalmitate, Sorbitan trioleate, N-Carbamoyl methoxypolyethylene glycol 2000-1,2-distearol, myristic acid, Steareth, Stearic acid, Polyoxyl 40 stearate, Sucrose stearate, Tocopherol, polyoxyl castor oil, Triglyceride synthetic, Trimyristin, Tristearin, magnesium stearate, lecithin, lauryl sulfate, Vitamin E, egg yolk phosphatides, docusate sodium, Polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (Macrogol-15 hydroxystearate ).

As used herein, a “PEG-vitamin E” means a compound of the formula:

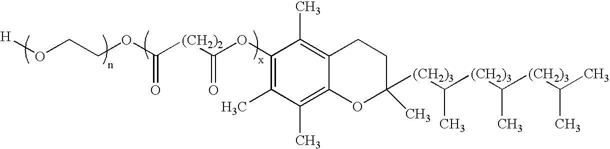

wherein the variable x is 0 or 1 and the variable n is about 1 to about 20,000, preferably, from about 3 to about 1000. Preferably, the PEG-vitamin E is α-tocopheryl polyethylene glycol 1000 succinate, referred to herein as tocophersolan (sold by Eastman Chemical Co. under the trade name vitamin E TPGS NF). In tocophersolan, x is 1 and n has an average value of 22. Other preferred PEG-vitamin Es, include tocophereth-5, tocophereth-10, tocophereth-12, tocophereth-18, and tocophereth-50. In such tocophereths, x is 0 and the average ethoxylation value is 5, 10, 12, 18, and 50 respectively. PEG-vitamin Es are available commercially, for example, from Eastman Chemical Co., Kingsport, Tenn. and Pacific Corporation, Seoul, Korea.

As used herein, the term “polysorbate” means a compound of the general formula:

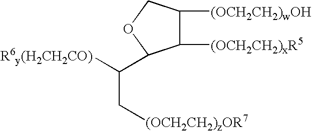

wherein the sum of W+X+Y+Z is an integer having an average value of about 5, 4, or 20; R⁵, R⁶, and R⁷ are independently H,

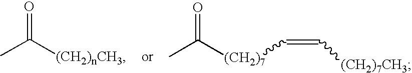

and n is an integer ranging from 8 to 20. Preferred polysorbates are polysorbate 20, 21, 40, 60, 61, 65, 80, 81, 85, more preferably polysorbate 20 or polysorbate 80. Polysorbates are available commercially under the trade name TWEEN from Rhône-Poulenc, Shelton, Conn.

The term “active” refers to a pharmaceutical, more specifically to paclitaxel, derivatives, and pharmaceutically acceptable salts thereof.

The term “cremophor” means PEG-35 castor oil (commercially available from BASF, Washington, N.J., under the trade name Cremophor® EL).

As used herein, the phrase “paclitaxel-free” refers to an oil, solvent, or surfactant of the invention which does not contain paclitaxel or a pharmaceutically acceptable derivative thereof.

As used herein, the phrase “ethanol-free” refers to a formulation which contains less than about 5% by weight ethanol, preferably less than about 3% by weight, more preferably less than about 2% by weight and most preferably less than about 1% by weight.

As used herein, the phrase “formulations of the invention” refers to a specific composition or combination of ingredients (i.e., one or more oils, solvents, and or surfactants and any other excipients, diluents, or carriers) useful for administering, delivering, or distributing paclitaxel, a derivative, or salt thereof. A “formulations of the invention” may or may not include an active. Preferably, the formulations of the invention are suitable for administration by the oral route either as a solid or as a solution or suspension. The formulations of the invention may also be sterile. A formulation of the invention can be in the form of a solid, liquid, semisolid, gel, suspension, emulsion, or a solution.

Solids include any solid form, such as a powder, a compressed pharmaceutical dosage form, or a lyophilized solid. In one embodiment, formulations of the invention if solid or other than liquid, are suitable for reconstitution into an oral formulation, such as a formulation of the invention.

The phrase “pharmaceutically acceptable salt(s)”, as used herein, means those salts of paclitaxel derivatives that retain the biological effectiveness and properties of the free acids or free bases and that are not otherwise unacceptable for pharmaceutical use. Pharmaceutically acceptable salts of paclitaxel derivatives include salts of acidic or basic groups which may be present in the paclitaxel derivatives. Derivatives of paclitaxel that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as chloride, bromide, iodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Derivatives of paclitaxel that include an amino moiety can also form pharmaceutically acceptable salts with various amino acids, in addition to the acids mentioned above. Derivatives of paclitaxel that are acidic in nature are capable of forming a wide variety of salts with various inorganic and organic bases. Suitable base salts are formed from bases that donate cations to form non-toxic salts, suitable cations include, but are not limited to, sodium, aluminum, calcium, lithium, magnesium, potassium, zinc and diethanolamine salts. For a review on pharmaceutically acceptable salts see Berge et al., J Pharm. Sci., 66, 1-19 (1977), incorporated herein by reference.

As used herein, the term “excipient” means the substances used to formulate actives into pharmaceutical formulations; in a preferred embodiment, an excipient does not lower or interfere with the primary therapeutic effect of the active. Preferably, an excipient is therapeutically inert. The term “excipient” encompasses carriers, diluents, vehicles, solubilizers, stabilizers, and binders. Excipients can also be those substances present in a pharmaceutical formulation as an indirect result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the phrase “formulation component” means any substance in addition to the active in a sample. Preferably, a formulation component is therapeutically inactive. Examples of optional formulation components, i.e., other than an oil, solvent or surfactant, include, but are not limited to, excipients, diluents, stabilizers, preservatives, colorants, flavoring agents, buffering agents, binders and combinations thereof.

As used herein the term “oral formulation” refers to a pharmaceutical formulation capable of being administered orally as discrete pharmaceutical unit dosage forms, each containing a predetermined amount of the active ingredients, such as capsules, cachets, soft elastic gelatin capsules, hard gelatin capsules, tablets, caplets, or aerosols sprays, as a powder or granules, or as a solution or a suspension in a non-aqueous liquid, an oil-in-water emulsion, or a water-in-oil liquid emulsion.

### 4. DETAILED DESCRIPTION OF THE INVENTION

### 4.1. Formulations of the Invention

The invention encompasses oral paclitaxel formulations which are both stable during storage and have increased bioavailability. The formulations of the invention are useful for orally administering paclitaxel, its derivatives, or pharmaceutically acceptable salts of such derivatives to patients in need thereof. The formulations of the invention are particularly useful for increasing the oral bioavailability of paclitaxel, such that the oral route is a useful route of administration.

More generally, the formulations of the invention or mixtures thereof can increase the solubility of paclitaxel and derivatives thereof as well as its absorption in the gastrointestinal system such that the invention encompasses an oral formulation which can be effectively used without relying on costly, inconvenient and potentially toxic intravenous formulations and administration.

In one embodiment, the invention concerns a pharmaceutical formulation suitable or oral administration to a mammal comprising:

(a) paclitaxel, a derivative, or a pharmaceutically acceptable salt thereof; and

(b) one or more of an oil, solvent, or surfactant each of which is defined further below.

In another embodiment, the invention concerns a pharmaceutical formulation suitable for oral administration to a mammal comprising:

(a) paclitaxel, a derivative, or a pharmaceutically acceptable salt thereof; and

(b) an oil, a solvent, and a surfactant.

In one embodiment, said pharmaceutical formulation comprises more than about 80 mg/mL of paclitaxel or a derivative thereof; in a preferred embodiment more than about 90 mg/mL; and in a more preferred embodiment about 100 mg/ml/mL of paclitaxel.

A few preferred non-aqueous liquid formulations of the invention (i.e., formulations 1-4) are shown below. Each formulation contains up to and preferably 100 mg/mL paclitaxel and optionally further contains 2 mg/mL citric acid.

| | | |
|---|---|---|
| | | Preferred volume |
| Component | Volume % range | % range |

Formulation 1

| | | |
|---|---|---|
| PEG-400 | 0.1% to 99% | 30% to 35% |
| Polysorbate 80 | 0.1% to 99% | 60% to 90% |
| Triacetin | 0% to 7% | 1% to 7% |

Formulation 2

| | | |
|---|---|---|
| PEG-400 | 0.1% to 99% | 55% to 65% |
| Polysorbate 80 | 0.1% to 99% | 25% to 35% |
| Triacetin | 0% to 7% | 0% to 7% |

Formulation 3

| | | |
|---|---|---|
| PEG-400 | 0.1% to 99% | 65% to 85% |
| Polysorbate-80 | 0.1% to 99% | 5% to 20% |
| Triacetin | 0% to 99% | 0% to 7% |

Formulation 4

| | | |
|---|---|---|
| Transcutol | 0.1% to 99% | 75% to 99.9% |
| PEG-Vitamin E | 0% to 59% | 0% to 25% |

The formulations of the invention can optionally include a pharmaceutically acceptable organic acid for stabilization of the formulation during storage and use. These organic acids include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, or acetic acid. Formulations of the invention preferably include citric acid or a hydrate thereof.

4.1.1. Oils

In the formulations of the invention, the oils include, but are not limited to Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil-hydrogenated soybean oil, Captex 335 (C8/C10 triglycerides from coconut oil), corn glycerides, corn oil, corn oil PEG-6 esters, cottonseed oil, Captex 200 (C8/C10 diesters of propylene glycol of coconut oil), diacetylated monoglycerides, Sesame oil, Soybean oil hydrogenated, Capmul MCM (C8/C10 mono-/diglycerides from coconut oil), Benzyl Benzoate, Soybean oil, olive oil, PEG vegetable oil, Vegetable oil, Vegetable oil hydrogenated, peanut oil, mineral oil, or Vegetable shortening and mixtures thereof. In a preferred embodiment, the oil is sesame oil, soybean oil, mineral oil or mixtures thereof.

In a preferred embodiment, the pharmaceutical formulation comprises about 0% to about 7% by volume of an oil.

It is within the skill of the art to utilize weight percent rather than volume percent by performing the appropriate conversion. The weight percents of the components of the invention may be determined by standard conversion from volume to weight using the appropriate density. However, it should be noted that the weight percents are approximately equal to the volume percents used herein, with the exception that when citric acid is added to the formulations discussed herein, an adjustment must be made.

4.1.2. Solvents

In another embodiment of the invention, the solvent includes, but is not limited to, ethanol, cetyl alcohol, glyceryl stearate, isopropyl alcohol, diethylarnine, ethylene glycol monoethyl ether, Transcutol, benzyl alcohol, glyceryl oleate, gelucire, myristyl alcohol, diethanolamine, glycerin, glyceryl distearate, gamma cyclodextrin, gelatin, ethylene glycol, polyethylene glycol 8000, Cresol, Propylene glycol, polyethylene glycol, polyethylene glycol 1000, polyethylene glycol 1450, polyethylene glycol 1540, polyethylene glycol 200, polyethylene glycol 300, polyethylene glycol 3350, polyethylene glycol 3500, Povidone, polyethylene glycol 400, polyethylene glycol 4000, polyethylene glycol 600, polyethylene glycol 6000, Stearyl alcohol, polyethylene glycol t-dodecylthioether, polyethylene oxide, Triacetin, Polyvinylpyridine, Polyvinyl alcohol, Polypropylene glycol, or Arlacel 186 (monoolein:propylene glycol=90:10) and mixtures thereof. In a preferred embodiment, the solvent is Triacetin, cresol, PEG-200, PEG-300, PEG-400, Transcutol, ethylene glycol monoethyl ethe or mixtures threeof.

In in a preferred embodiment, the pharmaceutical formulation comprises about 30% to about 85% by volume of solvent, for example about 30% to about 85% by volume of polyethyleneglycol-400, preferably about 30% to about 35% by volume polyethyleneglycol-400, about 55% to about 65% by volume polyethyleneglycol-400, or about 65% to about 85% by volume polyethyleneglycol-400.

It should be noted that more than one solvent may be used, for example, Triacetin may be used as a co-solvent from about 1% to about 7% by volume.

4.1.3 Surfactants

In still another embodiment of the invention, the surfactant includes, but is not limited to, Polyoxyl 20 stearate, Polyoxyl 35 castor oil, poloxamer, polyoxyethylene sorbitan monoisostearate, polyethylene glycol 40 sorbitan diisostearate, Polyoxyl 40 Hydrogenated castor oil, Polysorbate, Polysorbate 20, Polysorbate 40, Polyoxyl 60 stearate, Polysorbate 85, Polysorbate 60, poloxamer 331, polyoxyethylene fatty acid esters, Polyoxyl 40 castor oil, poloxamer 188, polyoxyethylene polyoxypropylene 1800, oleic acid, Sodium desoxycholate, Sodium lauryl sulfate, Sorbitan monolaurate, Sorbitan monooleate, Sorbitan monopalmitate, Sorbitan trioleate, N-Carbamoyl methoxypolyethylene glycol 2000-1,2-distearol, myristic acid, Steareth, Stearic acid, Polyoxyl 40 stearate, Sucrose stearate, Tocopherol, polyoxyl castor oil, Triglyceride synthetic, Trimyristin, Tristearin, magnesium stearate, lecithin, lauryl sulfate, Vitamin E, egg yolk phosphatides, docusate sodium, Polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (Macrogol-15 hydroxystearate ) and mixtures thereof. In a preferred embodiment, the surfactant is Labrasol, polysorbate 20, polysorbate 80, PEG-Vitamin E, cremophor or mixtures thereof.

In a preferred embodiment, the pharmaceutical formulation comprises about 5% to about 90% by volume of surfactant, for example, about 5% to about 90% by volume polysorbate 80, preferably about 60% to about 90% by volume polysorbate 80; or about 5% to about 25% by volume polysorbate 80.

In a more preferred embodiment, the pharmaceutical formulation comprises about 75% to about 100% by volume Transcutol and about 0% to about 25% by volume PEG-Vitamin E.

4.1.4. P-Glycoprotein Inhibitors

Alternatively, the formulations of the invention can be used with certain P-glycoprotein inhibitors which can be administered in a pre-dosage or post-dosage form to be used in combination of the formulation. These agents include, but are not limited to, cyclosporin, cyclosporin A, Gelucire 44/14, polysorbate 20, polysorbate 40, polysorbate 60, polysorbate 80, polysorbate 85. PEG-12 stearate, PEG-20 stearate, PEG-25 stearate, PEG-30 stearate, PEG-40 stearate, PEG-45 stearate, PEG-50 stearate, PEG-100 stearate, PEG-40 hydrogenated castor oil, PEG-35 castor oil and Solutol HS.

### 4.2. Bioavailability and Methods of Use

The invention encompasses oral paclitaxel formulations which have improved solubility over previous attempts to make an oral paclitaxel formulation. See e.g. Malingré et. al. British Journal of Cancer 2001, 85(10), 1472-1477. The formulations of the invention are taken up by the cells of the gastrointestinal tract after dissolution and passage through the gastric fluid. In a related embodiment, the invention encompasses oral paclitaxel formulations which are free of cremophor. The invention encompasses formulations that precipitate in the gastrointestinal system into particles of a size suitable for an increase in the dissolution profile of paclitaxel in gastric media. In a related embodiment, formulations of the invention remain dissolved in gastric fluid for absorption, or preferably precipitate into discrete particles which measure less than about 10 μm in size; that is, the particles are of a size which increases the solubility and dissolution rate of paclitaxel in the gastrointestinal system. Preferably, the formulation has a particle size of less than about 5 μm in size, more preferably less than about 1 μm in size, and still more preferably less than about 600 nm in size.

The formulations of the invention are useful for the oral delivery of paclitaxel, derivatives or pharmaceutically acceptable salts thereof to mammals in need thereof. The formulations of the invention are useful for treating or preventing mammalian cancers and other medical conditions treatable by paclitaxel. By “treating” it is meant that the formulations are administered to inhibit or reduce the rate of cancer-cell proliferation in an effort to induce partial or total remission, for example, inhibiting cell division by promoting microtubule formation. For instance, the formulations of the invention are useful for treating solid tumors and blood-born tumors. Examples of cancers treatable or preventable by formulations of the invention include, but are not limited to, cancers of the lymph node; breast; cervix; uterus; gastrointestinal tract; lung; ovary; prostate; mouth; brain; head; neck; throat; testes; kidney; pancreas; bone; spleen; liver; bladder; skin; larynx; nasal passages; AIDS-related cancers, and cancers of the blood. The formulations can be used alone or in combination with other chemotherapeutics. The mode, dosage, and schedule of administration of paclitaxel, derivatives, and pharmaceutically acceptable salts thereof in human cancer patients have been extensively studied, see, e.g., 1989 Ann. Int. Med., 11:273-279, incorporated herein by reference. The doses can readily be determined by in vitro tests and/or in vivo tests. Doses and dose frequency will vary with the disease and severity thereof, patient age, height and can be monitored or adjusted by the clinician. The amount of paclitaxel to be administered is in accordance with the recommended doses found in the Physicians Desk Reference adjusted for the bioavailability by oral route.

### 4.3. Preparation of Oral Paclitaxel Formulations

The formulations of the invention can be prepared by combining the actives, oils, solvents, and surfactants of the invention, and other components using well-known pharmaceutical-formulation methods. Formulation of liquid dosage forms, is described in Remington: the Science and Practice of Pharmacy, Alfonso R. Gennaro ed., Mack Publishing Co. Easton, Pa., 19th ed., 1995, Chapters 87 and 88; incorporated herein by reference. A comprehensive discussion on formulating solid forms, such as powders, tablets, pills, and capsules is presented in Remington: the Science and Practice of Pharmacy, Alfonso R. Gennaro ed., Mack Publishing Co. Easton, Pa., 19th ed., 1995, Chapters 91 and 92, incorporated herein by reference. A comprehensive discussion on formulating solutions, emulsions, and suspensions is presented in Remington: the Science and Practice of Pharmacy, Alfonso R. Gennaro ed., Mack Publishing Co. Easton, Pa., 19th ed., 1995, Chapter 86, incorporated herein by reference. Formulations of the invention in the form of gels and semisolids containing the active can be prepared according to well known methods. For instance, by mixing the active with the paclitaxel solubilizers of the invention, and any additional components or excipients in a standard V-blender. Preferably, solids, liquid concentrates, semisolids, gels, suspensions, and emulsions, contain about 25 milligrams to about 2500 milligrams of active, more preferably, about 50 milligrams to about 500 milligrams.

In one process, the formulations of the invention are prepared by dissolving paclitaxel, a derivative or a pharmaceutically acceptable salt thereof in a solvent of the invention prior to dilution with one or more paclitaxel-free oils, solvents, or surfactants as described herein. Such a method increases the amount of paclitaxel that can be formulated and thus delivered orally, as well as affects the particle size of the precipitate that forms upon contact with gastric fluid and gastrointestinal media. In a preferred embodiment, the solvent used in this process is PEG-400 or Transcutol or mixtures thereof. In another preferred embodiment, the concentration of paclitaxel after dilution with one or more paclitaxel-free oils, solvents, or surfactants is greater than 80 mg/mL, more preferably greater than 90 mg/mL, most preferably 100 mg/mL. In still another preferred embodiment, said paclitaxel-free oils, solvents, or surfactants include, but are not limited to, Triacetin, Transcutol or polysorbate 80.

### 4.4. Dosage Forms and Combination Therapies

The invention encompasses single-unit dosage forms and multi-unit dosage forms of paclitaxel, derivatives, and pharmaceutically acceptable salts thereof in solid, liquid, semisolid, gel, suspension, emulsion, or solution form. In one embodiment, the invention relates to single-unit dosage and multi-unit dosage forms. In another embodiment, the invention relates to single-unit dosage and multi-unit dosage forms of solids, semi-solids, and gels. Preferably, the formulations are easy to handle, stable for storage and shipment, and inexpensive to manufacture compared to previous paclitaxel formulations. The oral solutions, suspensions or emulsions may also be contained within a suitable unit dosage form such as a soft elastic gelatin capsule, a hard gelatin capsule or as a oral solution.

The formulations of the invention can include additional pharmaceutically acceptable excipients. Preferred additional excipients do not affect the stability or oral bioavailability of the formulations. Examples of suitable excipients, such as binders and fillers are listed in Remington: the Science and Practice of Pharmacy, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000.

Oral formulations of the invention are preferably in the form of capsules, tablets, pills, soft-gel capsules, hard-gel capsules, emulsions, solutions, or suspensions. Formulations of the invention can optionally include one or more sweetening agents and one or more flavoring agents to provide a pharmaceutically palatable preparation. A therapeutically effective oral dosage for formulations of the invention is determined by standard clinical techniques according to the judgment of a medical practitioner. For example, in addition to information provided in medical reference books and pharmaceutical literature, well-known in vitro or in vivo assays can be used to help identify optimal dosages.

Paclitaxel, as well as all oils, solvents, and surfactants of the invention, and any other formulation components are commercially available or can be synthesized as desired and are preferably purified prior to use in accordance with good manufacturing procedure.

4.4.1. Oral Dosage Forms

Pharmaceutical compositions of the invention that are suitable for oral administration can be presented as discrete dosage forms, such as, but are not limited to, tablets (e.g., chewable tablets), caplets, capsules, soft eleastic gelatin capsules, hard gelatin capsules and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets, caplets, hard gelatin capsules and soft elastic gelatin capsules represent the most advantageous oral dosage unit forms. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Fourmulations of the invention may also be formulated in a soft elastic gelatin capsule unit dosage form by using conventional methods well known in the art. See, e.g., Ebert, Pharm. Tech., 1(5):44-50 (1977). Soft elastic gelatin capsules have a soft, globular gelatin shell somewhat thicker than that of hard gelatin capsules, wherein a gelatin is plasticized by the addition of plasticizing agent, e.g., glycerin, sorbitol, or a similar polyol. The hardness of the capsule shell may be changed by varying the type of gelatin used and the amounts of plasticizer and water. The soft gelatin shells may contain a preservative, such as methyl- and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycerides, surfactants, such as polysorbates, or a combination thereof.

In another example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-5 81, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-58 1. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

4.4.2. Controlled Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.5 Combination Therapy

The present formulations can include additional actives and thus can serve as base formulation for combination therapy. Such additional actives can be included and distributed in the formulation itself or administered adjunctively with the formulation. Additional actives can be any other cancer and cancer-related pharmaceuticals, such as cisplatin, carboplatin, epirubicin, leuprolide, bicalutamide, goserelin implant, irinotecan, gemcitabine, and sargramostim; cardiovascular drugs; such as amlodipine besylate, enalapril maleate, losartan potassium lisinopril, irbesartan, nifedipine, diltiazem, clopidogrel, digoxin, abciximab, furosemide, amiodarone, beraprost, and tocopheryl; anti-infective agents, such as amoxicillin, clavulanate, ciprofloxacin, azithromycin, itraconazole, acyclovir fluconazole, terbinafine, erythromycin, and sulfisoxazole acetyl; psychotherapeutic agents, such as fluoxetine, paroxetine, sertaline, vanlafaxine, bupropion, olanzapine, alprazolam, methylphenidate, fluvoxamine, and ergoloid; gastrointestinal medicaments, such as omeprazole, lansoprazole, ranitidine, famotidine, ondansetron, granisetron, sulfasalazine, and infliximab; respiratory therapies, such as loratadine, fexofenadine, cetirizine, fluticasone, salmeterol xinafoate, and budesonide; cholesterol reducers, such as simvastatin, atorvastatin calcium, pravastatin, lovastatin, bezafibrate, ciprofibrate, and gemfibrozil; blood modifiers, such as epoetin alpha, enoxaparin, and antihemophilic factor; antiarthritic agents, such as celecoxib, diclofenac sodium, nabumetone, misoprostol, and rofecoxib; AIDS and AIDS-related drugs, such as larnivudine, zidovudine, indinavir, stavudine, and lamivudine; diabetes and diabetes-related therapies, such as metformin, troglitazone, and acarbose; biologicals, such as hepatitis vaccines; Hormones, such as estradiol; immunosuppressive agents, such as cyclosporine, mycophenolate mofetil, and methylprednisolone; analgesics, such as tramadol, fentanyl, metamizole, ketoprofen, morphine, lysine acetylsalicylate, ketoralac tromethamine, morphine, loxoprofen sodium, and ibuprofen; dermatological products, such as isotretinoin and clindamycin; anesthetics, such as propofol, midazolarn, and lidocaine; migraine therapies, such as sumatriptan succinate, zolmitriptan, and rizatriptan; sedatives and hypnotics, such as, zolpidem, triazolam, and hycosine butylbromide; multiple sclerosis agents, such as interferon beta-1a, interferon beta-1a, and glatiramer; osteoporosis agents, such as vitamin k₂; cystic fibrosis agents, such as dornase alpha and tobramycin; Alzheimer's disease therapies, such as dolasetron and donepezil; and imaging agents, such as iohexol, technetium Tc99m sestamibi, iomeprol, gadodiamide, ioversol, and iopromide; or pharmaceutically acceptable salts thereof.

Paclitaxel and the other therapeutics agent can act additively or, more preferably, synergistically. In a preferred embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent.

In a specific embodiment, the invention encompasses methods of orally adiministering paclitaxel comprising administering to a patient in need thereof, an oral paclitaxel formulation, and an additional therapeutic agent. In a further embodiment, the invention encompasses methods of orally administering paclitaxel comprising administering to a patient in need thereof, an oral paclitaxel formulation, an additional therapeutic agent, and a p-glycoprotein inhibitor.

The present invention will be further understood by reference to the following non-limiting examples. The following examples are provided for illustrative purposes only and are not to be construed as limiting the invention scope of the invention in any manner.

### 5. EXAMPLES

### 5.1 Example 1

### Particle Size Studies

Upon dilution in the gastrointestinal tract, the paclitaxel must either stay in solution, or “precipitate” in the form that has higher solubility or faster rate of dissolution than the “parent” form of the paclitaxel. Further, particle sizes less than 10 μm are preferred (more preferably less than 5 μm, and still more preferably less than about 600 nm) for uptake in the gastrointestinal system and thus are desired for any precipitated formed by administration of the oral formulations of the invention.

The resulting particle characteristics for these formulations in the gastrointestinal tract has been simulated by 500-fold dilution into simulated gastric fluid (SGF). The dilution solutions are visually inspected and both the particle sizes and size distribution are measured by dynamic laser light scatting (Zetasizer™ 2000, Malvern Instruments, Inc.) This highly sensitive analytical instrument can determine particle size in a range of 2-3000 nm. The particle size data for the formulations diluted in SGF are listed in Table 1.

TABLE 1
Particle sizes of Formulations A & B
diluted in simulated gastric fluid.
Formulation A (Contains 100 mg/mL Paclitaxel)

| | | | Particle size at | |
|---|---|---|---|---|
| | | | 1:500 Dilution in | |
| Triacetin | PEG-400 | Tween 80 | SGF | |
| 1%-7% | 30%-35% | 60%-90% | 8-30 | nm |
| 0%-7% | 55%-65% | 25%-35% | 300-600 | nm |
| 0%-7% | 65%-85% | 5%-20% | 1-5 | μm |

Formulation B (Contains 100 mg/mL Paclitaxel)

| | | Particle size in |
|---|---|---|
| | | 1:500 Dilution in |
| Transcutol | PEG-Vitamin E | SGF |
| 75-100% | 0-25% | 300 nm-10 μm |

These studies suggest that the particle size stability in diluent SGF is affected by the formulation preparation method; when formulation A is prepared by dissolving paclitaxel first in PEG-400, followed by addition of Triacetin and Tween 80 (without paclitaxel) in the amounts listed in Table 3, the physical stability of the diluted mixture is better than a formulation prepared by mixing the 3 components together which each contain paclitaxel in the individual solutions. As shown in the particle size results of formulation A in Table 1, different ratios of the same components can result in different particle sizes upon dilution in SGF.

### 5.2 Example 2

### Citric Acid Stability Studies

A one-month short-term stability study of above formulations has been carried out. Citric acid as a stabilizer is included in this study at different concentration. The results of this study suggest that paclitaxel can be stabilized by adding citric acid at 2.0 mg/mL to all the oral formulations. Table 2 is the summary of results.

TABLE 2

| | | Storage | Percentage | Percentage |
|---|---|---|---|---|
| | Citric | condition | of | of |
| | Acid | (° C./% | degradants | degradants |
| Formulation | (mg/mL) | humidity) | at initial | at 1 month |
| Formulation A | 0 | 25° C./60% | 0.12 | 0.68 |
| | 1.0 | 25° C./60% | 0.13 | 0.62 |
| | 2.0 | 25° C./60% | 0.13 | 0.37 |
| Formulation B | 0 | 25° C./60% | 0.12 | 1.36 |
| | 1.0 | 25° C./60% | 0.13 | 0.05 |
| | 2.0 | 25° C./60% | 0.13 | 0.10 |
| Formulation A | 0 | 40° C./75% | 0.12 | 2.12 |
| | 1.0 | 40° C./75% | 0.13 | 0.71 |
| | 2.0 | 40° C./75% | 0.13 | 0.36 |
| Formulation B | 0 | 40° C./75% | 0.12 | 8.23 |
| | 1.0 | 40° C./75% | 0.13 | 0.11 |
| | 2.0 | 40° C./75% | 0.13 | 0.12 |

Adding 2.0 mg/mL citric acid has no effect on paclitaxel solubility and the particle size after diluted into simulated gastric fluid. As a result of this stability study, 2.0 mg/mL citric acid can be included and is preferably included in all the oral paclitaxel formulations of the invention.

### 5.3. Example 3

### P-Glycoprotein Inhibition

P-glycoprotein inhibition has been determined with an in vitro assay. Evaluation of the existing literature suggested that excipients with a polyethylene glycol (PEG) polymeric segment afford P-gp inhibiting characteristics to the molecule. Therefore, the initial pool of compounds screened included excipients that were acceptable for oral or IV use and included PEG in the chemical structure. Compounds were first screened for toxicity in the NIH3T3 cell line and then tested for P-gp inhibition with paclitaxel using the protocol outlined below:

Day 0: Seed cells and incubate.

Day 1: Add potential P-gp inhibitor at fixed concentration and paclitaxel at various concentrations and incubate for 2 days.

Day 3: Add MTT and extraction/lysis buffer.

Day 4: Read absorbance to measure cell growth. Calculate IC50 to determine amount of paclitaxel required for cell death

After calculations of IC50 values, relative rank order of the compounds were determined. Final results of single-excipient screens, in which oral excipients are ranked according to P-gp inhibition (measured by an IC50 ratio>1.0), and include, but are not limited to: Gelucire 44/14; polysorbate 20; polysorbate 40; polysorbate 60; polysorbate 80; polysorbate 85; PEG-12 stearate; PEG-20 stearate; PEG-25 stearate; PEG-30 stearate; PEG-40 stearate; PEG-45 stearate; PEG 50 stearate; PEG-100 stearate; PEG-40 hydrogenated castor oil; PEG-35 castor oil; and Solutol HS. This list represents components that will be pre-dosed or administered concurrently with the paclitaxel formulation.

### 5.4. Example 4

### In Vivo Pharmacokinetic Studies

Two formulations of the present invention with the following compositions were prepared and used for pharmacokinetic studies in rats:

Formulation 1: 83 mg/mL paclitaxel dissolved in 4.9% Triacetin, 34.0% PEG 400, 58.0% Polysorbate 80, 3.0% water.

Formulation 2: 100 mg/mL paclitaxel dissolved in 75% Transcutol, and 25% vitamin E TPGS.

A 20 mg/kg oral dose was given to male Sprague-Dawley rats for all groups. For the rats treated with cyclosporin A, they were pre-treated with 5 mg/kg cyclosporin A 1 hour before dosing, and treated again with 5 mg/kg cyclosporin A immediately before dosing of the various formulations. Three male Sprague-Dawley rats (7 weeks old, averaged 250 g each) from Charles River Japan were used per experimental group. Plasma samples were collected from tail vein at 0.083, 0.5, 1, 2, 3, 4, and 8 hrs post-dosing and stored at −20° C. until assayed with HPLC.

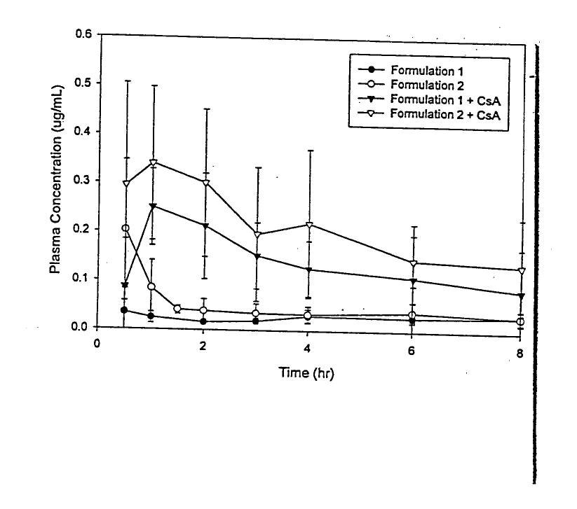

The pharmacokinetic profiles for each of the samples and the control are illustrated in FIG. 1. The results indicate that Formulation 1 and 2 can result in measurable improved bioavailability in rats when the p-glycoprotein inhibitor Cyclosporin A was used. In addition, Formulation 2 demonstrated higher bioavailability over Formulation 1. This can be explained by the fact that Formulation 2 results in nanoparticles with a smaller particle size (volume average diameter 147nm) than Formulation 1 (volume average diameter 269 nm) when the formulations are diluted into simulated gastric fluids. These data confirm that smaller particles exhibit faster rate of dissolution and therefore result in improved bioavailability in vivo.

The foregoing has demonstrated the pertinent and important features of the present invention. One of skill in the art will be appreciate that numerous modifications and embodiments may be devised. Therefore, it is intended that the appended claims cover all such modifications and embodiments.

What is claimed is:

1. A pharmaceutical formulation suitable for oral administration to a mammal comprising:
(a) paclitaxel, a derivative, or a pharmaceutically acceptable salt thereof; and
(b) one or more of an oil, solvent, or surfactant wherein said formulation is a non-aqueous liquid.

2. A pharmaceutical formulation suitable for oral administration to a mammal comprising:
(a) paclitaxel, a derivative, or a pharmaceutically acceptable salt thereof; and
(b) one or more of an oil, solvent, or surfactant wherein said formulation is in the form of a solid, semisolid, gel, suspension, or emulsion.

3. The pharmaceutical formulation of claim 1, wherein the formulation is adapted for delivery as a capsule, soft elastic gelatin capsule, hard gelatin capsule, caplet, aerosol, spray, solution, suspension or an emulsion.

4. The pharmaceutical formulation of claim 2, wherein the formulation is adapted for delivery as a cachet, tablet, capsule, soft elastic gelatin capsule, hard gelatin capsule, caplet, aerosol, powder or granules.

5. The pharmaceutical formulation of claim 3 or 4, wherein the formulation is adapted for delivery as a soft elastic gelatin capsule.

6. The pharmaceutical formulation of claim 3 or 4, wherein the formulation is adapted for delivery as a hard gelatin capsule.

7. The pharmaceutical formulation of claim 3 or 4, wherein the formulation is adapted for delivery as an oral solution.

8. The pharmaceutical formulation of claim 1 or 2, wherein the formulation is in single-unit-dosage form.

9. The pharmaceutical formulation of claim 1 or 2, wherein the formulation is in multi-unit-dosage form.

10. The pharmaceutical formulation of claim 1 or 2, further comprising additional excipients, flavoring agents or preservatives.

11. The pharmaceutical formulation of claim 1 or 2, comprising more than about 80 mg/mL of paclitaxel.

12. The pharmaceutical formulation of claim 11, comprising more than about 90 mg/mL of paclitaxel.

13. The pharmaceutical formulation of claim 12, comprising 100 mg/mL of paclitaxel.

14. The pharmaceutical formulation of claim 1 or 2, further comprising an organic acid selected from the group consisting of ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, acetic acid and hydrates thereof.

15. The pharmaceutical formulation of claim 14, wherein said organic acid is citric acid.

16. The pharmaceutical formulation of claim 15, wherein said citric acid is present in an amount of about 2 mg/mL.

17. The pharmaceutical formulation of claim 1 or 2, wherein said oil is Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil--hydrogenated soybean oil, Captex 335 (C8/C10 triglycerides from coconut oil), corn glycerides, corn oil, corn oil PEG-6 esters, cottonseed oil, Captex 200 (C8/C10 diesters of propylene glycol of coconut oil), diacetylated monoglycerides, Sesame oil, Soybean oil hydrogenated, Capmul MCM (C8/C10 mono-/diglycerides from coconut oil), Benzyl Benzoate, Soybean oil, olive oil, PEG vegetable oil, Vegetable oil, Vegetable oil hydrogenated, peanut oil, mineral oil, Vegetable shortening, or mixtures thereof.

18. The pharmaceutical formulation of claim 1 or 2, wherein said solvent is ethanol, cetyl alcohol, glyceryl stearate, isopropyl alcohol, diethylamine, ethylene glycol monoethyl ether, Transcutol, benzyl alcohol, glyceryl oleate, gelucire, myristyl alcohol, diethanolamine, glycerin, glyceryl distearate, gamma cyclodextrin, gelatin, ethylene glycol, polyethylene glycol 8000, Cresol, Propylene glycol, polyethylene glycol, polyethylene glycol 1000, polyethylene glycol 1450, polyethylene glycol 1540, polyethylene glycol 200, polyethylene glycol 300, polyethylene glycol 3350, polyethylene glycol 3500, Povidone, polyethylene glycol 400, polyethylene glycol 4000, polyethylene glycol 600, polyethylene glycol 6000, Stearyl alcohol, polyethylene glycol t-dodecylthioehter, polyethylene oxide, Triacetin, Polyvinylpyridine, Polyvinyl alcohol, Polypropylene glycol, Arlacel 186 (monoolein:propylene glycol=90:10) or mixtures thereof.

19. The pharmaceutical formulation of claim 1 or 2, wherein said surfactant is Polyoxyl 20 stearate, Polyoxyl 35 castor oil, poloxamer, polyoxyethylene sorbitan monoisostearate, polyethylene glycol 40 sorbitan diisostearate, Polyoxyl 40 Hydrogenated castor oil, Polysorbate, Polysorbate 20, Polysorbate 40, Polyoxyl 60 stearate, Polysorbate 85, Polysorbate60, poloxamer 331, polyoxyethylene fatty acid esters, Polyoxyl 40 castor oil, poloxamer 188, polyoxyethylene polyoxypropylene 1800, oleic acid, Sodium desoxycholate, Sodium lauryl sulfate, Sorbitan monolaurate, Sorbitan monooleate, Sorbitan monopalmitate, Sorbitan trioleate, N-Carbamoyl methoxypolyethylene glycol 2000-1,2-distearol, myristic acid, Steareth, Stearic acid, Polyoxyl 40 stearate, Sucrose stearate, Tocopherol, polyoxyl castor oil, Triglyceri,de synthetic, Trimyristin, Tristearin, magnesium stearate, lecithin, lauryl sulfate, Vitamin E, Vitamin E TPGS, egg yolk phosphatides, docusate sodium, Polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), and Solutol (Macrogol-15 hydroxystearate ), or mixtures thereof.

20. The pharmaceutical formulation of claim 1 or 2, comprising about 0% to about 7% by volume of an oil.

21. The pharmaceutical formulation of claim 1 or 2, comprising about 30% to about 85% by volume of a solvent.

22. The pharmaceutical formulation of claim 1 or 2, comprising about 5% to about 90% by volume of a surfactant.

23. The pharmaceutical formulation of claim 21, comprising about 30% to about 85% by volume polyethyleneglycol-400.

24. The pharmaceutical formulation of claim 23, comprising about 30% to about 35% by volume polyethyleneglycol-400.

25. The pharmaceutical formulation of claim 24, comprising about 55% to about 65% by volume polyethyleneglycol-400.

26. The pharmaceutical formulation of claim 25, comprising about 65% to about 85% by volume polyethyleneglycol-400.

27. The pharmaceutical formulation of claim 22, comprising about 5% to about 90% by volume polysorbate 80.

28. The pharmaceutical formulation of claim 27, comprising about 60% to about 90% by volume polysorbate 80.

29. The pharmaceutical formulation of claim 28, comprising about 5% to about 25% by volume polysorbate 80.

30. The pharmaceutical formulation of claim 21, comprising about 75% to about 100% by volume Transcutol.

31. The pharmaceutical formulation of claim 22, comprising about 0% to about 25% by volume PEG-Vitamin E.

32. The pharmaceutical formulation of claim 1 or 2, comprising about 1% to about 7% by volume of triacetin.

33. The pharmaceutical composition of claim 1, wherein said paclitaxel precipitates into discrete particles in gastric fluid having particle size of less than about 10 μM.

34. The pharmaceutical composition of claim 33, wherein said discrete particles have a particle size of less than about 5 μm.

35. The pharmaceutical composition of claim 34, wherein said discrete particles have a particle size of less than about 1 μm.

36. The pharmaceutical composition of claim 35, wherein said discrete particles have a particle size of less than about 600 nm.

37. The pharmaceutical formulation of claim 1 or 2, which are ethanol-free.

38. The pharmaceutical formulation of claim 1 or 2, which does not comprise cremophor.

39. A method of orally delivering paclitaxel, a derivative or a pharmaceutically acceptable salt thereof to a mammal in need thereof, comprising administration of:
(a) paclitaxel, a derivative, or a pharmaceutically acceptable salt thereof; and
(b) one or more of an oil, solvent, or surfactant in a non-aqueous liquid, wherein said paclitaxel remains dissolved in gastric fluid for absorption or precipitates into discrete particles upon dilution with gastric fluid.

40. A method of orally delivering paclitaxel, a derivative or a pharmaceutically acceptable salt thereof to a mammal in need thereof, comprising administration of:
(a) paclitaxel, a derivative, or a pharmaceutically acceptable salt thereof; and
(b) one or more of an oil, solvent, or surfactant in a solid, semisolid, gel, suspension, or emulsion.

41. The method of claim 39 or 40, wherein said mammal is a human.

42. The method of claim 39, wherein said discrete particles have a particle size of less than about 10 μm.

43. The method of claim 42, wherein said discrete particles have a particle size of less than about 5 μm.

44. The method of claim 43, wherein said discrete particles have a particle size of less than about 1 μm.

45. The method of claim 44, wherein said discrete particles have a particle size of less than about 600 nm.

46. A method of formulating a pharmaceutical composition comprising dissolving paclitaxel, a derivative or a pharmaceutically acceptable salt thereof in a solvent chosen from polyethylene glycol-200 (PEG-200), polyethylene glycol-300 (PEG-300), polyethylene glycol-400 (PEG-400), diethylene glycol monoethyl ether (Transcutol), or ethylene glycol monoethyl ether, prior to dilution with one or more paclitaxel-free oils, solvents, or surfactants.

47. The method of claim 46 wherein said solvent is PEG-400 or Transcutol.

48. The method of claim 47 wherein the concentration of paclitaxel after dilution with one or more paclitaxel-free oils, solvents, or surfactants is more than 80 mg/mL.

49. The method of claim 48 wherein the concentration of paclitaxel after dilution with one or more paclitaxel-free oils, solvents, or surfactants is more than 90 mg/mL.

50. The method of claim 49 wherein the concentration of paclitaxel after dilution with one or more paclitaxel-free oils, solvents, or surfactants is 100 mg/mL.

51. The method of claim 46 wherein said paclitaxel-free oils, solvents, or surfactants are triacetin, Transcutol or polysorbate 80.

52. The method of claim 46 wherein said formulation is administered before during or after the administration of a P-glycoprotein inhibitor.

53. A single unit dosage form suitable for oral administration to a human which comprises:
(a) paclitaxel, a derivative or a pharmaceutically acceptable salt thereof;
(b) one or more of an oil selected from the group consisting of sesame oil, soybean oil, and mineral oil;
(c) one or more of a solvent selected from the group consisting of triacetin, cresol, PEG-200, PEG-300, PEG-400, transcutol, and ethylene glycol monoethyl ether;
(d) one or more of a surfactant selected from the group consisting of labrasol, polysorbate 20, polysorbate 80, PEG-Vitamin E and cremophor; and
(e) an organic acid.

54. A method for orally delivering paclitael, a derivative or a pharmaceutically acceptable salt thereof to a human in need thereof which comprises:
(a) administering an oral formulation of paclitaxel comprising
(i) paclitaxel, a derivative, or a pharmaceutically acceptable salt thereof, and
(ii) one or more of an oil, solvent, or surfactant in a non-aqueous liquid,; and
(b) administering a P-glycoprotein inhibitor before, during or after step (a).

55. A method for orally delivering paclitael, a derivative or a pharmaceutically acceptable salt thereof to a human in need thereof which comprises:
(a) administering an oral formulation of paclitaxel comprising
(i) paclitaxel, a derivative, or a pharmaceutically acceptable salt thereof, and
(ii) one or more of an oil, solvent, or surfactant in a solid, semisolid, gel, suspension, or emulsion.
(b) administering a P-glycoprotein inhibitor before, during or after step (a).

56. The method of claim 54 or 55 wherein the P-glycoprotein inhibitor is Gelucire 44/14, polysorbate 20, polysorbate 40, polysorbate 60, polysorbate 80, polysorbate 85, PEG-12 stearate, PEG-20 stearate, PEG-25 stearate, PEG-30 stearate, PEG-40 stearate, PEG-45 stearate, PEG 50 stearate, PEG-100 stearate, PEG-40 hydrogenated castor oil, PEG-35 castor oil, Solutol HS, or a cyclosporin.

57. The method of claim 56 wherein the P-glycoprotein inhibitor is a cyclosporin.

58. The method of claim 57 wherein the cyclosporin is cyclosporin A.
